(19) United States
(12) Patent Application Publication
(10) Pub. No.: US 2007/0239234 A1
(43) Pub. Date: Oct. 11, 2007

(54) THERAPEUTIC TREATMENT APPARATUS

(76) Inventors: Jan Henning Simonsen, Struer (DK); Peter Bjerring, Risskov (DK)

Correspondence Address: KOHN & ASSOCIATES, PLLC, 30500 NORTHWESTERN HWY, STE 410, FARMINGTON HILLS, MI, 48334, US

(21) Appl. No.: 11/739,135

(22) Filed: Apr. 24, 2007

Related U.S. Application Data
(63) Continuation-in-part of application No. 10/473,672, filed on Oct. 30, 2003.
(63) Continuation-in-part of application No. 10/520,515, filed on Jan. 7, 2005.

(30) Foreign Application Priority Data
Mar. 30, 2001 (DK) PA 2001-00550
Jul. 8, 2002 (DK) PA 2002-01075

(51) Int. Cl. A61N 5/06

(52) U.S. Cl. 607/88

(57) ABSTRACT

The apparatus for non-invasive treatment of skin disorders or removal of unwanted hair comprises a reflector unit having therein at least one light source for emitting incoherent light, the reflector unit and the light source being replaceable after activation thereof and disposable after such replacement. The apparatus includes an energy source for providing an electrical current to the light source or light sources, an on/off switch for selectively triggering supply of the electrical current to the light source or light sources and preventing said current supply; and a controller for controlling at least one of the intensity, the pulse length and the sequence of each light source, at least one of the light sources being adapted to emit a series of light pulses.

### CROSS-REFERENCE TO RELATED APPLICATIONS

This application is a Continuation-In-Part of U.S. patent application Ser. No. 10/473,672, filed Oct. 30, 2003, which is a National Phase of International Application No. PCT/DK01/00799, filed Nov. 30, 2001, which claims priority to Denmark Patent Application Serial No. PA 2001-00550, filed Mar. 30, 2001; and also of U.S. patent application Ser. No. 10/520,515, filed Jan. 7, 2005, which is a National Phase of International Application No. PCT/DK03/00349, filed May 27, 2003, which claims priority to Denmark Patent Application Serial No. PA 2002-01075, filed Jul. 8, 2002, all of which are incorporated herein by reference.

### TECHNICAL FIELD

The present invention concerns therapeutic treatment apparatus for non-invasive treatment of skin disorders such as discoloration, acne, wrinkles, visible blood vessels, cellulite and stretch marks, or for removal of unwanted hair, softening of scar tissue and the like.

### BACKGROUND OF THE INVENTION

It is known to use electromagnetic radiation in therapeutic and cosmetic medical applications such as treatment of skin disorders. For example, U.S. Pat. No. 4,298,005 to Mutzhas describes a continuous ultraviolet lamp with cosmetic, photobiological, and photochemical applications. Treatment based on using the UV portion of the spectrum and its photochemical interaction with skin is described. The power delivered to the skin using Mutzhas' lamp is described as 150 WVm², which does not have a significant effect on skin temperature.

Lasers have also been used for dermatological procedures, including Argon lasers, CO₂ lasers, Nd(Yag) lasers, copper vapor lasers, ruby lasers and dye lasers. For example, US patent No. 4,829,262 to Furumoto, describes a method of constructing a dye laser used in dermatology applications. Two skin conditions which may be treated by laser radiation are external skin irregularities such as local differences in the pigmentation or structure of the skin, and vascular disorders lying deeper under the skin which cause a variety of skin abnormalities including port wine stains, telangiectasias, leg veins and cherry and spider angiomas. Laser treatment of such skin disorders generally includes localized heating of the treatment area by absorption of the radiation. Heating the skin changes or corrects the skin disorder causing the full or partial disappearance of the skin abnormality.

Certain external disorders such as pigmented lesions can also be treated by heating the skin very fast to a high enough temperature in order to evaporate parts of the skin. Deeper-lying vascular disorders are more typically treated by heating the blood to a high enough temperature to cause it to coagulate. The disorder will then eventually disappear. To control the treatment depth a pulsed radiation source is often used. The depth the heat penetrates in the blood vessel is controlled by controlling the pulse wave bandwidth of the radiation source. The absorption and scattering coefficients of the skin also affect the heat penetration. These coefficients are a function of the constituents of skin and the wavelength of the radiation. Specifically, the absorption coefficient of light in the epidermis and dermis tends to be a slowly varying, monotonically decreasing function of wavelength. Thus, the wavelength of the light should be conditioned according to the vessel size being treated.

The effectiveness of lasers for applications such as tattoo removal and removal of birth and age marks is diminished because lasers are monochromatic. A laser of a given wavelength may be effectively used to treat a first type of skin pigmentation disorder, but, if the specific wavelength of the laser is not absorbed efficiently by skin having a second type of disorder, it will be ineffective for the second type of skin disorder. Also lasers are usually complicated, expensive to manufacture, large in comparison to the amount of power delivered, unreliable and difficult to maintain.

The wavelength of the light also affects vascular disorder treatment because blood content in the vicinity of the vascular disorders varies, and blood content affects the absorption coefficient of the treatment area. Oxyhemoglobin is the main chromophore which controls the optical properties of blood and has strong absorption bands in the visible region. More particularly, the strongest absorption peak of oxyhemoglobin occurs at 418 nm and has a bandwidth of 60 nm. Two additional absorption peaks with lower absorption coefficients occur at 542 and 577 nm. The total bandwidth of these two peaks is in the order of 100 nm. Additionally, light in the wavelength range of 500 to 550 nm is desirable for the treatment of blood vessel disorders of the skin since it is absorbed by the blood and penetrates through the skin. Longer wavelengths up to 1000 nm are also effective since they can penetrate deeper into the skin, heating the blood vessel by thermal conductivity. Also, longer wavelengths are effective for treatment of larger diameter vessels because the lower absorption coefficient is compensated for by the longer path of light in the vessel.

Especially when treating vascular disorders in the vicinity of the skin surface, it was found during development of the present apparatus that treatment in the red spectrum turned the hemoglobin instantly into oxyhemoglobin. Oxyhemoglobin is easily detectable as a black coloring. As this takes place in the entire area treated and as it spreads out with the bloodstream and furthermore lasts for a period of time, this effect is undesirable.

### OBJECT OF THE INVENTION

Accordingly, a wide band electromagnetic radiation source covering the near UV and the visible region of the spectrum, and optionally the near infra-red, is desirable for treatment of external skin and vascular disorders. The range of wavelengths of the light source should be sufficient to optimize treatment for any of a number of applications. Such a therapeutic radiation apparatus should also be capable of providing an optimal wavelength range within the overall range for the specific disorder being treated. The intensity of the light should be sufficient to cause the required internal and/or external thermal effect by raising the temperature in or around the treatment area to the required temperature, but without causing pain or skin problems. Also, the pulse width should be variable over a wide enough range so as to achieve optimal penetration depth for each application.

Therefore, it is desirable to provide therapeutic treatment apparatus comprising one or more light sources having a wide range of wavelengths, which can be selected according to the required skin treatment, with a controlled pulse band width and a high enough energy density for application to and in the affected area. The term “therapeutic” as used herein also encompasses cosmetic applications, namely where no specific medical benefit is achieved using the apparatus according to the invention.

### SUMMARY OF THE INVENTION

The present invention therefore provides therapeutic treatment apparatus for non-invasive treatment of skin disorders or removal of unwanted hair, which apparatus comprises a reflector unit having therein at least one light source for emitting incoherent light, the reflector unit and the light source being replaceable after activation thereof and disposable after such replacement.

The apparatus according to the invention further includes
i) an energy source for providing an electrical current to the light source;
ii) an on/off switch for selectively triggering supply of said electrical current to the light source and preventing the current supply; and
iii) means for controlling the intensity and/or pulse length and/or sequence of each of the light sources, at least one of the light sources being arranged to emit a series of light pulses.

Some types of skin treatment as mentioned above require that the light energy can be transmitted to a certain depth under the skin in order to be active in the pigmented layers of the skin if discoloration or the like are to be treated or the light should penetrate deeper to the fat layers of the skin in instances where the treatment is to avoid and/or mitigate stretch marks, cellulite or acne

Also for stimulating the skin's collagen content and production it is necessary to direct light energy to these specific areas at specific wavelengths in the skin, such that the collagen production will be stimulated. Increased collagen production helps the skin to remain flexible and to smooth out wrinkles or delay the formation of wrinkles. By combining different types of light sources, it is possible to provide variable intensities at different specific wavelengths and thereby optimize the treatment.

Furthermore, tests have indicated that the body's ability to produce collagen can be stimulated by introducing energy into the skin tissue. As the collagen helps in achieving a more supple tissue (which improves healing of scars and also prevents acne and other skin disorders), this is a desirable feature. By stressing the capillary blood vessels almost to the damage level during a cycle of treatments, the collagen production can be greatly improved. Therefore, treatment with apparatus according to the invention with an appropriate filter can improve skin healing. The treatment may be carried out both before and after scar formation.

The reflector unit preferably includes a concave reflector arranged proximate to the light source such that it can reflect the light in a general direction towards the treatment area. The arrangement of a reflector around the light source results in the advantages that the light is collected and guided in the general direction of the surface to be treated. By this arrangement, more of the emitted light energy can be used and thereby it is possible to operate the apparatus at lower energy levels, which makes it safer to use.

In an especially advantageous embodiment of the invention, the reflector has a plurality of concave surface indentations, which serve to collect and distribute the emitted light. By arranging a multitude of these indentations evenly on the reflector, the resulting emitted light from the light source will be of a more even character because the multitude of indentations will diffuse the light evenly over an opening in the apparatus.

The apparatus is preferably such that each light source can be controlled independently of the other light sources. It is thus possible to design a sequence of different light flashes such that during the treatment, the sequence of flashes is designed in order to optimize the effect of the delivered energy to the skin.

For most treatments, the amount of energy delivered to the skin is decisive on the successful treatment. Especially when removing unwanted hair or treating other disorders, it is important to deliver the maximum amount of energy in a very short time span, such that the energy intensity is maximized. On the other hand, it is also important to limit the amount of delivered energy such that unwanted side effects such as pain burns, discoloration arising from the light treatment etc. can be avoided.

In this connection, in apparatus according to the present invention it has been found that by rapidly flashing a single light source or a series of light sources rapidly one after the other, the energy intensity in the zone which has to be treated can be greatly increased without significant risk of side effects. It thus becomes possible to carry out an effective treatment without side effects.

Disposable flash bulbs in a modified form can be advantageously used in apparatus according to the invention. The traditional disposable flash bulbs can be designed to have the required light pulse length as mentioned above from 15 milliseconds to about 2 seconds; an electronic flash is much faster.

As traditional flash light bulbs are comparable to normal light bulbs in that the light is emitted in an even distribution to the surroundings, it can for the purpose of the present invention be advantageous to collect the emitted light and guide it towards the area which is to be treated. This can in an advantageous embodiment of the invention be done by arranging a prism or optical block between the light source and the object to be treated such that the optical block collects and guides the emitted light to the object to be treated.

By providing such an optical block in order to collect and thereby concentrate the light emitted from the disposable light source on to the surface of the treatment area, it is possible to carry out the treatment with a lower energy consumption as more light is collected and transmitted on to the treatment zone than would be expected, if the light was just transmitted freely to the surroundings.

In a further advantageous embodiment of the invention the optical block has two further important uses. One of these is to keep the light source at a fixed distance from the treatment surface. By giving the optical block a certain thickness it is possible to assure that there is a certain distance between the surface to be treated and the light source.

In practice, this is done by having a disposable light source with a rather extended optical block which is adapted to be touching the skin during treatment. Hereby the optical block acts as a kind of spacer in order to be able to control the precise energy delivery to the treatment area.

The second function of the optical block is as a safety measure. By making the disposable light source stronger in the direction which is intended to be in contact with the treatment area, it is less likely that a disposable light source would fracture in the lens and thereby hurt the patient who is being treated. This is important when using traditional flash bulbs, as they are usually made of rather thin glass; by having a material placed within the bulb which is ignited and thereby explodes, it can fracture and shatter because of faults in the glass. Any ignitable material can cause explosions which are too powerful for the rather thin glass to withstand which then can result in fractured glass.

In a still further advantageous embodiment a filter can be arranged such that the emitted light will travel through the filter. In this way it is possible to select the optimum band of wavelengths which will have the most effective treatment according to the disorder which is to be treated. For example, it is known that water absorbs energy at the wavelength 1015 nm. In order to avoid overheating in the skin it is desirable to avoid pulsing too much light energy at this wavelength into the skin, whereby overheating due to water's absorption of the energy can be avoided, and therefore a more efficient treatment of the disorder can be achieved instead of spending the energy on heating the skin's content of water. A similar relationship is applicable for treating vascular disorders such as blood vessels immediately under the skin or for treating rosacea (which is an acne-like disease, often in central parts of the face).

For treatment of these kinds of disorders it is interesting to be able to use specific wavelengths for treating the blood vessels under the skin, and for stimulating collagen production.

In a still further advantageous embodiment, a different filter may be arranged with respect to every distinct light source, so that it is possible to design the light treatment for a specific purpose.

As the light influences the skin's condition, especially in the outermost layers of the skin, the effective characteristics of the skin also changes. This in turn influences the effectiveness of the light treatment in that the skin's ability to absorb the transmitted energy varies according to the wavelength of the energy source and the composition of the tissue. Some of these changes in the skin characteristics are almost instantaneous.

In an embodiment of the invention, a filter comprising different sections/areas is used. Each area/section filters light in a specific wavelength range and is moved across the light source during the light emitting sequence. By moving the appropriate filter at the right velocity across the light source, it is possible to transmit a high and constant level of energy into the tissue, even when the tissue's characteristics concerning absorption ability and reflectivity changes. Depending on the skin type, type of treatment and selected light emitting means/sequence, the filter can be designed accordingly as the changes in the skin tissue are known.

By combining the above mentioned features with each light source being controlled independently with respect to intensity, pulse length, wavelength, time delay between pulses, the relevant flashes can be fully controlled according to the optimum treatment.

A special problem arises when treating darker skin. These skin types typically have a high content of melanin, the pigment ingredient in skin. Also, darker skin types usually have darker hair. For treating these skin and hair types, more energy needs to be delivered to the skin. Thus, there is a danger of causing burns, pain or other forms of irritation. These skin types can advantageously he preheated up to 50-60° C. or even 80° C. before light treatment. Temperature means, for example, an infrared thermometer, may be arranged and, optionally, connected to a control circuitry in the apparatus for controlling the skin and/or tissue temperature in the treatment area.

Tests have indicated that it is the difference in temperature within the skin which causes damage to the tissue. Therefore, by preheating the area around the treatment area, the temperature difference as well as the temperature gradient between unheated tissue and the treatment area can be minimized while at the same time it is possible to deliver enough energy into the desired skin layer or hair follicle for successful treatment

In still another embodiment of the invention between four and ten light sources are arranged in a reflector unit and an optical element such as a lens or block is arranged in front of the reflector in the reflector unit. This embodiment is especially advantageous in that first of all the apparatus may be a single unit, namely the reflector unit closed off by the optical element, but also by the necessary light sources in the same unit. A complete treatment can be facilitated by such a unit. The optical element can be designed either alone or in cooperation with the reflector to target the emitted light at one well defined target area to be treated.

In practice, the incoherent light sources are preferably selected from flash bulbs and flash tubes, such as electronic flash tubes.

The energy delivered to the skin is desired to be at least around 2 joules per cm² per flash (sometimes up to 25 joules per cm² per flash). This energy density should be high enough to have an effect on the skin disorders described above, but not enough to cause damage to normal skin disorders or skin types. By combining different filters and selecting different intensities it is possible to take into consideration the type of skin to be treated i.e. fair skin should have higher intensity than darker skin. The treatment apparatus can be applied in a number of applications for either therapeutic treatment or cosmetic treatment. By selecting and/or combining different light sources with different wavelengths and intensities, designing these in a specific sequence and pulse length it is possible to design light treatment for the skin disorders mentioned above. For removal of hair it is possible to first pulse or heat up the hair follicle, overlay the initial heating with a stronger light pulse whereby the hair sac will be destroyed.

One specific example of how to design a hair removal treatment would be to first ignite a flash device and then within 100 or 200 milliseconds, ignite a second flash device and thereafter follow up by a more powerful light source for destroying the hair sac. Additionally, a third flash device could be ignited in order for the affected skin to cool down slower. In this fashion it is possible to combine and alter the sequence between flash bulb devices and electronic flashes completely freely according to optimum treatment.

In preferred apparatus according to the invention, a plurality of the light sources is arranged in one replaceable/disposable unit. This is an especially advantageous feature of the present invention in that the apparatus itself includes the energy source and the trigger or switch mechanism; the latter can be used for multiple treatments for multiple disorders by simply selecting the appropriate light source(s) for a specific treatment. The apparatus according to the invention can be designed as a mobile handheld device whereby treatment can be carried out anywhere. Only the light source and reflector unit has to be replaced.

Furthermore, by knowing about the energy levels required in order to treat fair skin in comparison to dark skin it is possible to design the disposable apparatus according to the invention such that the user will be able to acquire complete treatments, especially tailored for their type of skin.

As indicated, the light sources are arranged in a replaceable/disposable reflector unit. Once the light source has been activated and spent, it can be disposed of very easily by replacing the entire bulb comprising one or more light sources and installing a replacement unit either for treating the same kind of skin disorder or treating a different kind of skin disorder as described above.

The apparatus according to the invention includes means for controlling the sequence and/or the pulse length, and/or intensity of each light emitting source. By incorporating control means, for example, in the form of a microchip and microswitches, it is possible to initiate different light sources at different times whereby a multitude of different treatments can be carried out according to the programming of the control means.

Tests have shown that electronic flash tubes comparable to those used in disposable cameras proved to be effective enough in order to be able to provide apparatus according to the present invention.

Tests have shown that by designing the light emission by varying the intensity, the pulse length and the wavelength, it is also possible to have an effect on the treatment of psoriasis as well as birthmarks

In connection with plastic surgery and especially for the healing of scars after surgical invasions, it has shown that the light treatment which stimulated the collagen production improves the healing process and minimizes scar tissue, whereby the traces of either plastic surgery or regular surgery will be minimized. The treatment can also be carried out before the surgery in order to prepare the skin in the effected area to be more flexible and stimulative and with a high collagen content.

In one example of a treatment using apparatus according to the invention, visible blood veins present in the lower leg area were treated. First, a series of “warming up” shots by an electronic flash device were directed against the area to he treated. With the prototype it is possible to treat an area of approximately up to 1600 mm² in one cycle. After initial heating of the area, an energy intense flash of about 2 J/cm² was directed at the treatment area. The process which is accelerated or initiated in the skin is that in the warming up phase the blood is heated and a “blue response” phenomenon appears. This is due to the hemoglobin comprising oxygen is deoxidized whereby the blue color appears in the area. During exposure to the actual treatment flash dose the hemoglobin in the blood in the treatment area loses an oxygen molecule and hooks up with a Fe-molecule, whereby so-called met-haemoglobin is created. This is detected as a black coloration in the treatment area. The blood will coagulate and created what look like a severe bruise (black and blue). As the veins are destroyed, the normal blood circulation in the area will transport the met-haemoglobin away from the area, which after a period of time will regain its normal skin tone.

### BRIEF DESCRIPTION OF THE DRAWINGS

Exemplary embodiments of the present invention will now be explained with reference to the accompanying drawing, in which

FIG. 1 illustrates an example of apparatus according to the invention;

FIG. 2a illustrates a light source having more ignition stages for use in apparatus according to the invention;

FIG. 2b illustrates another light source having multiple ignition stages,

FIG. 2c illustrates another embodiment of a light source having multiple ignition stages;

FIG. 3 illustrates in schematic form an example of apparatus according to the invention; and

FIG. 4 illustrates the spacing using a prism or optical block in apparatus according to the invention.

### DETAILED DESCRIPTION OF THE INVENTION

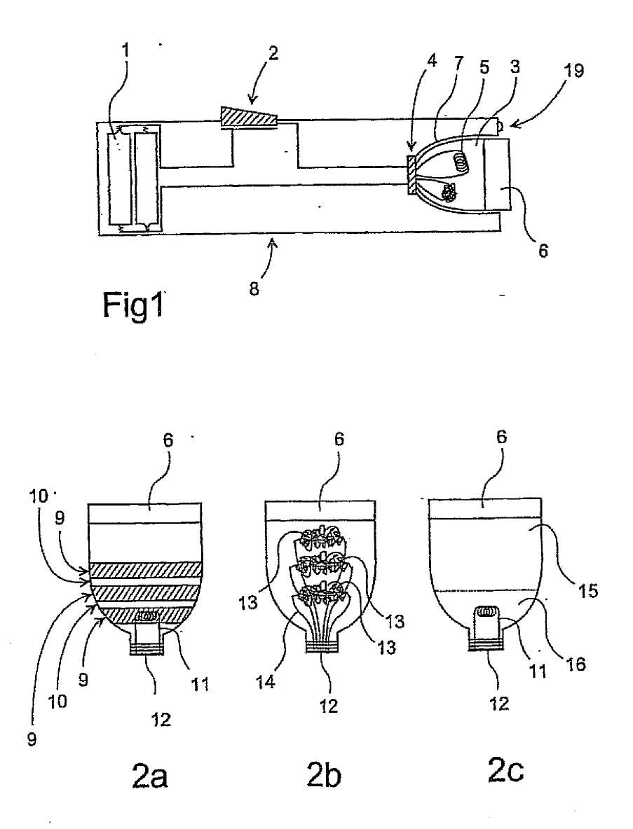

In FIG. 1 is illustrated self-contained apparatus according to the invention, which can in some aspects be compared to a common flash light. Batteries 1 represent the electrical energy source and a trigger switch 2 is mounted on a conductor connecting the batteries to a disposable incoherent light source 3. The disposable light source is in this embodiment a disposable flash bulb, which is mounted in a socket 4.

Before each use, a new suitable flash bulb, that is to say an incoherent light source 3 is mounted in the socket 4. When the switch makes the electrical contact, current passes to the light source 3, whereby ignitable material 5 is ignited and emits light. In this embodiment of the invention a light source having an optical block 6 mounted in front of the light source is illustrated. Part of the emitted light from the ignition of the material 5 passes straight through the prism or optical block 6 and into the treatment area. The rest of the light is bounced off the reflector 7 and guided in a comparable manner to a head light on a car out through the prism or optical block 6 and into the treatment area. By this arrangement substantially all the light emitted by ignition of ignitable material 5 will be guided towards the treatment area on a patient. This allows for the relatively low energy levels used in the apparatus, which makes it safer for the patient to use.

In these examples, the prism or optical block is an integral part of the disposable unit. The prism or optical block can however be a part of the remainder of the apparatus, which makes the apparatus according to the invention simpler, and thereby cheaper.

All the above mentioned components of the apparatus are in this embodiment of the invention arranged in a housing or casing 8. The housing or casing can be made from any suitable material, such as, for example metals or plastics.

In FIGS. 2a, 2b and 2c, three different embodiments of a suitable incoherent light source are illustrated. In FIG. 2a a light source, wherein a layered structure of ignitable materials 9, separated by delaying fuses 10 is illustrated. Furthermore, the ignition device 11 is seen placed in the lowermost layer of ignitable material. The lowermost part of the light source is equipped with a footing 12, which is adapted to be mounted in the socket 4 of the apparatus illustrated in FIG. 1. In front of the ignitable materials seen in the direction of mounting the light source in the apparatus in FIG. 1 a prism or optical block 6 is arranged.

In FIG. 2b, a similar device having a footing 12 and prism or optical block is illustrated. Instead of layered ignitable materials the ignitable material in the embodiment shown in FIG. 2b is in the form of metal meshes 13 with separate electrodes 14, whereby circuitry arranged in the device shown in FIG. 1 will control the ignition of the separate meshes according to a pre-scheduled light pulse programme.

The light source illustrated in FIG. 2c consists of the footing 12 and a prism or optical block 6 and an ignition device 11. The interior of the light source in this embodiment is illustrated as having two separate chambers 15, 16. It is envisaged that two different gasses can be provided in the chambers 15, 16, whereby once the first gas in chamber 16 is ignited an initial flash will appear which will also ignite the gas in chamber 15. In this manner a two stage light pulse will be achieved.

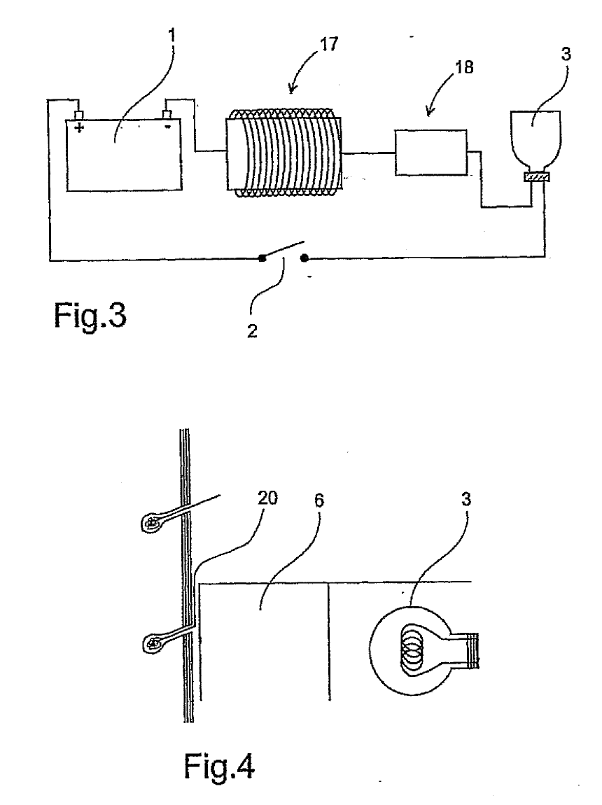

FIG. 3 shows a schematic representation of the apparatus of FIG. 1. The energy source 1 is connected to a transformer 17, which controls the current in the circuitry and the pulse sent to the light source 3. This is particularly important where a multi-stage lighting apparatus is required.

In FIG. 4 use of apparatus according to the invention is illustrated. The light source 3 is illustrated as a traditional single stage light bulb, but can be any of the other light sources described above. The prism or optical block 6 arranged in front of the light source also has the function of spacing. By giving the prism or optical block a certain thickness a minimum distance will at all times be kept between the light source and the surface of the skin to be treated. This assures that the correct level of light energy can be transmitted on to the treatment surface, whereby burns or other damage arising from too intense a treatment can be avoided. By arranging the prism or optical block as a plurality of separate elements, the light will be evenly distributed to the surface 20 of the prism or optical block, whereby a larger surface can be treated with uniform optical energy.

What is claimed is:

1. Therapeutic treatment apparatus for non-invasive treatment of skin disorders or removal of unwanted hair, which apparatus comprises a reflector unit having therein at least one light source for emitting incoherent light, the reflector unit and the light source being replaceable after activation thereof and disposable after such replacement, the apparatus further including
i) an energy source for providing an electrical current to said at least one light source;
ii) an on/off switch for selectively triggering supply of said electrical current to said light source and preventing said current supply; and
iii) means for controlling at least one of the intensity, the pulse length and the sequence of each said light source, at least one said light source being adapted to emit a series of light pulses.

2. Apparatus according to claim 1, including a plurality of said light sources and electrical circuitry such that each one of said light sources can be controlled independently of the others.

3. Apparatus according to claim 1, which includes a filter arranged to filter the light emitted from said at least one light source.

4. Apparatus according to claim 1, which contains four to ten of said light sources

5. Apparatus according to claim 1, wherein said light source for emitting incoherent light is selected from the group consisting of flash bulbs and electronic flash tubes.

6. Apparatus according to claim 1, wherein the reflector unit includes a concave reflector

7. Apparatus according to claim 6, wherein said reflector has a plurality of concave markings on a reflective surface thereof.

8. Apparatus for treatment of skin or removal of unwanted hair, which apparatus comprises:
a) a housing having an opening in a face thereof and a cavity connected to said opening;
b) at least one incoherent light source for emitting incoherent light with wavelengths between 550 nanometers and 1050 nanometers, said light source being within the housing and being disposable after use;
c) at least one concave reflector arranged in said cavity remote from said opening so as to direct light from said light source through said opening towards the skin of a patient to be treated,
d) an optical block spanning said opening between said at least one light source and said skin, said block being arranged to direct light from said at least one light source to said skin and spacing said light source from said skin;
d) a filter between said block and said light source,
e) an energy source for providing an electrical current to said at least one light source;
f) an on/off switch for selectively triggering supply of said electrical current to said light source or preventing said current supply; and
g) means for pulsing light from said light source.

9. Apparatus according to claim 7, which includes a plurality of said light sources and electrical circuitry for controlling a parameter selected from intensity, pulse length and sequence of operation of each one of said light sources independently of the other ones of said light sources.

10. Apparatus according to claim 8, which includes a plurality of said light sources, each having a corresponding said reflector.

11. Apparatus according to claim 9, wherein each said light source comprises an electronic flash tube.

12. Apparatus according to claim 9, wherein said plurality of said incoherent light sources are arranged in a single replaceable and disposable unit including said corresponding reflectors.

13. Apparatus according to claim 7, wherein the concave reflector has a plurality of concave markings on a reflective surface thereof.

14. Apparatus according to claim 7, wherein each said light source comprises an electronic flash tube.
